(19) United States
(12) Patent Application Publication
(10) Pub. No.: US 2008/0213357 A1
(43) Pub. Date: Sep. 4, 2008

(54) Plant Derived Lipid Useful for Nutraceutical and Cosemeceutical Applications

(76) Inventors: Andrew Bruce Hebard, Winston-Salem, NC (US); Keith Coupland, South Cliffe (GB); David Neal Boughton, Colchester (GB); Marc Surette, Moncton (CA)

Correspondence Address: JONDLE & ASSOCIATES P.C., 858 HAPPY CANYON ROAD SUITE 230, CASTLE ROCK, CO, 80108, US

(21) Appl. No.: 12/014,628

(22) Filed: Jan. 15, 2008

Related U.S. Application Data
(60) Provisional application No. 60/889,459, filed on Feb. 12, 2007.

(51) Int. Cl. A61K 9/48; A61K 36/30

(52) U.S. Cl. 424/456; 424/776

(57) ABSTRACT

The present disclosure relates to novel nutritional and cosmeceutical compositions comprising an oil extracted from the seeds of Boraginaceae, particularly Buglossoides arvensis, in the range of from 1 to 100% by weight of the total composition. The oil has particular advantages over oils extracted from other seeds in that the oil is comprised of from 14% to at least 17% stearidonic acid and less than 7% gamma-linolenic acid. The oil composition is particularly suited for oral or topical administration and for dietary, cosmetic, pharmaceutical and healthcare uses.

### REFERENCE TO RELATED APPLICATIONS

This application claims priority under 35 U.S.C. § 119 to U.S. Application Ser. No. 60/889,459, filed Feb. 12, 2007 which is incorporated herein in its entirety by reference.

### BACKGROUND OF THE INVENTION

The present invention relates to a new and distinctive oil with high levels of stearidonic acid (SDA) and low levels of γ-linolenic acid (GLA) and to the compositions and uses comprising such oil. All publications cited in this application are herein incorporated by reference.

Triesters of glycerol are known as triglycerides or triacylglycerols. If the triglyceride is solid at room temperature, then it is generally considered to be a fat, whereas if it is a liquid at room temperature, then it is generally considered to be an oil. Most triglycerides in animals are fats, while most triglycerides in vegetables tend to be oils. Fatty acids can be obtained from these fats or oils by hydrolysis. Certain fatty acids, called essential fatty acids, must be present in the mammalian diet and are used in the body to synthesize, for example, prostaglandins. There are two main families of essential fatty acids: one is called the n-3 family (also known as the omega-3 or ω-3 family); and the other is called the n-6 family (also known as the omega-6 or ω-6 family). The two parent members of these families are α-linolenic acid (ALA, C18:3n-3) and linoleic acid (LA, C18:2n-6). Under normal circumstances in the mammalian body, both of these essential fatty acids are capable of being metabolized to longer chain polyunsaturated fatty acids (PUFAs) by a series of enzyme mediated reactions; however both ALA and LA cannot be produced in the body and therefore must be obtained in the diet. The desaturation and elongation pathways for the n-3 and n-6 PUFAs are shown in FIG. 1.

The health benefits of dietary PUFAs of the omega-3 family are well documented and generally accepted. Whelan, J., et. al., (2006) “Innovative sources of n-3 fatty acids” Annu. Rev. Nutr. 26:75-103. Two of the omega-3 PUFAs, eicosapentaenoic acid (EPA) and docosahexaenoic acid (DHA) are commonly found in marine oils from oily fish and also in certain microbial oils and are particularly valuable in reducing the risk of cardiovascular disease. Calder, P. C., et al. (2004) “Long chain n-3 fatty acids and cardiovascular disease further evidence and insights” Nutrition Res. 24:761-72. Furthermore, they are inversely related to the risk of sudden death among men with no prior evidence of cardiovascular disease. In addition, there is emerging evidence that omega-3 PUFAs may be of value in combating diabetes and age related dementia, such as Alzheimer's disease. See Horrobin, D. F., (1993) “Fatty acid metabolism in health and disease: The role of delta-6 desaturase” Am. J. Clin. Nutr. 57(suppl): 732S-737S; Barre, D. E., (2007) “The role of consumption of alpha-linolenic, eicosapentaenoic and docosahexaenoic acids in human metabolic syndrome and type 2 diabetes—a mini review” J. Oleo. Sci. 56(7): 319-325; and Norris, J. M., et al., (2007) Omega-3 polyunsaturated fatty acid intake and islet autoimmunity in children at increased risk for type 1 diabetes. JAMA. September 26;298(12): 1420-1428. Consumption of fish oil containing omega-3 fatty acids has been shown to decrease the risk of incident Alzheimers disease. Arch. Neurol. 60(7): pp. 940-946. Stearidonic acid (SDA), an essential fatty acid, is also a polyunsaturated fatty acid of the n-3 family. Chemically, it can be described as 6c,9c,12c,15c-octadecatetraenoic acid (C18:4n-3) and is a precursor to EPA and docosapentaenoic acid (DPA, C22:5n-3). Other essential fatty acids include ALA and GLA (C18:3n-6). SDA is found in small quantities in marine oils, such as sardine oil and in a small number of vascular plants such as blackcurrent (Ribes nigrum) and Echium (Echium plantageneum) and in lipids isolated from micro-organisms. More recently, SDA has been genetically engineered into canola and soybean. Ursin, V. M. (2003) “Modification of plant lipids for human health: development of functional land-based omega-3 fatty acids” J. Nutr. 133:4271-4274.

SDA is also a transient product formed (produced) in the mammalian body by the desaturation of ALA with the enzyme delta-6 desaturase. EPA is formed from SDA by elongation and further desaturation. The enzymes responsible for these reactions are also used by other substrates, such as GLA for elongation and desaturation. Therefore, there exists a competition between SDA and GLA for these enzymes.

Delta-6 desaturase is cited as the rate limiting step in fatty acid metabolism. See Horrobin, D. F., (1993), Fatty acid metabolism in health and disease: The role of delta-6 desaturase. Am. J. Clin. Nutr. 57(suppl): 732S-737S. The activity of this enzyme is known to be of lower activity or down-regulated following certain illnesses, old age, poor diet and certain lifestyles. This is significant, because if the activity of delta-6 desaturase is lowered, then the body's capacity to make SDA (and the other compounds in the scheme shown in FIG. 1) is also lowered. One way to overcome this problem is with nutritional intervention by taking a dietary or nutritional supplement containing SDA, which is a substrate for EPA.

From the foregoing, a need exists for a cost-effective, renewable source of SDA-containing oil. Plant-derived SDA oils are a renewable resource and provide a viable alternative to marine oils (fish oils) as a source of EPA by biochemical conversion in the body.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

According to this invention, there is provided a rich natural, non-toxic, renewable source of SDA found in the oil of seeds of Buglossoides arvensis, also known as Lithospermum arvensis.

In another aspect, this invention provides for an oil that itself can be used in nutritional, cosmetic, personal care, pet care, aquaculture and pharmaceutical or healthcare products.

In another aspect, this invention provides for an oil that can be used with or without the need for additional treatment or purification and thus conferring advantages over purified SDA.

In another aspect, the invention provides for the use of an oil extracted from seeds of Buglossoides arvensis in topical application to, or oral ingestion by, the human or animal body. This oil can be used alone for these purposes or, preferably, it is used to form a part of a composition e.g. for topical application to, or oral ingestion by, the human or animal body.

In another aspect, this invention provides for the oil of Buglossoides arvensis to be delivered, whether oral or topically, in coated particles that are suitable for solubilizing or containing a wide variety of materials, including materials sensitive to physical, chemical or biological deterioration.

In another aspect, the oil of the present invention provides for a nutraceutical, cosmeceutical or pharmaceutical composition that may be orally or topically delivered in a hard capsule, a soft gel capsule, a liquid, a solid, a semi-solid, gel or powder.

In another aspect, the present invention provides for a nutraceutical composition with nutritional and health benefits to either or both, humans and animals.

In another aspect, the present invention provides for a cosmeceutical composition that provides for a better appearance, the well-being, the maintenance and health to either or both, humans and animals.

In another aspect, the present invention provides for a pharmaceutical composition with nutritional and health benefits to either or both, humans and animals.

In another aspect, because the oil has a low percentage of GLA and a high percentage of SDA there is a reduction in competition for the enzymes involved in converting SDA to EPA and hence a more efficient production of EPA in the body.

In another aspect, this invention provides for an increase in the total yield of an oil containing high levels of SDA and low levels of GLA by using the seeds of Buglossoides.

In another aspect, this invention provides for an increase in the total yield of an oil containing high levels of SDA and low levels of GLA by using the seeds of Buglossoides arvensis.

In another aspect, this invention provides for a more efficient method of producing an oil with high levels of SDA and low levels of GLA.

In another aspect, this invention provides for a more efficient and economical production of bulk oil from the seeds of the Buglossoides arvensis relative to other plant species.

In another aspect, this invention provides for a more economical benefit of producing an oil with high levels of SDA and low levels of GLA by using the seeds of Buglossoides.

In another aspect, this invention provides for a more economical benefit of producing an oil with high levels of SDA and low levels of GLA by using the seeds of Buglossoides arvensis.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Aquaculture. Aquaculture refers to the farming or cultivation of aquatic organisms, including fish, molluscs, crustaceans and aquatic plants in a controlled environment for business, governmental or personal (hobby) purposes. Farming implies some form of intervention in the rearing process to enhance production, such as regular stocking, feeding, protection from predators, etc.

Cosmetic. Cosmetic refers to preparations, such as topical powders, creams or gels designed to beautify the body by direct application.

Cosmeceutical. Refers to cosmetic products that have drug-like benefits, such as anti-aging creams and moisturizers.

Delta-6 desaturase. Refers to an enzyme which introduces a double bond between carbons six and seven from the carboxyl end of a fatty acid molecule.

Desaturase. Refers to a polypeptide which can desaturate one or more fatty acids to produce a mono- or polyunsaturated fatty acid or precursor thereof.

Dietary supplement. Refers to a food product designed to deliver specific nutrients with therapeutic effects and biological activity to the consumer of the product in dose-regulated portions via various delivery methods.

Essential fatty acid. Refers to a particular PUFA that an individual must ingest in order to survive, being unable to synthesize the particular essential fatty acid de novo. For example, mammals can not synthesize the essential fatty acid linoleic acid (18:2, ω-6). Other essential fatty acids include GLA (ω-6), DGLA (ω-6), ARA (ω-6), EPA (ω-3), DPA (ω-3) and DHA (ω-3).

Fat. Refers to a lipid substance that is solid at 25° C. and usually saturated.

Fatty acids. Refers to long chain aliphatic acids (alkanoic acids) of varying chain length, from about C₁₂ to C₂₂ (although both longer and shorter chain-length acids are known). The predominant chain lengths are between C₁₆ and C₂₂. The structure of a fatty acid is represented by a simple notation system of “X:Y”, where X is the total number of carbon atoms in the particular fatty acid and Y is the number of double bonds.

Food product. Refers to any food or feed suitable for consumption by humans, non-ruminant animals or ruminant animals or aquatic organisms. The food product may be a prepared and packaged food (e.g., mayonnaise, salad dressing, bread, or cheese food) or an animal feed (e.g., extruded and pelleted animal feed or coarse mixed feed). Additionally, the food product may be specifically for, but not limited to, companion animals, livestock, and fish.

Foodstuff. Refers to any substance fit for human or animal consumption.

Functional food. Refers to a food product to which a biologically active supplement has been added.

Gel. Gel refers to a colloid in which the disperse phase has combined with the dispersion medium to produce a semi-solid material, such as a jelly.

Geriatric food. Refers to a food product formulated for persons of advanced age (e.g. those persons of 65 years of age or older).

Infant food. Refers to a food product formulated for an infant such as milk formula.

Lipid. Refers to any element of the nutritional formula characterized by being a fatty acid or a derivative or substances related biosynthetically or functionally to these compounds including oils, fats, waxes and combinations thereof.

Liquid. Refers to the state of matter in which a substance exhibits a characteristic readiness to flow, little or no tendency to disperse and relatively high incompressibility.

Medical food. Refers to a food which is formulated to be consumed or administered by mouth, (tube feeding), any way internally, enterally, under the supervision of a physician, and/or qualified health administrator, and which is intended for the specific dietary management of a disease or condition for which distinctive nutritional requirements based on recognized scientific principles are established by medical evaluation.

Nutraceutical. Refers to any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. This definition also includes a bio-engineered designer vegetable food, rich in antioxidant ingredients, and a stimulant functional food or pharma-food

Nutritional. Refers to the process of nourishing or being nourished, whereby a living organism assimilates food and uses it for growth and for replacement of tissues.

Nutritional supplement. Refers to a food product formulated as a dietary or nutritional supplement to be used as part of a diet.

Oil. Refers to a lipid substance that is liquid at 25° C. and usually (poly) unsaturated.

Oral delivery vehicle. Refers to any means of delivering a pharmaceutical orally, including, but not limited to, capsules, pills, tablets and syrups.

Pet food/Companion food. Any article or component used as food, drink or for nutritional purposes by animals.

Pharmaceutical. Refers to any product or use pertaining to medical or pharmacy products.

Physiologically acceptable carrier. Refers to any carrier or excipient commonly used with oily pharmaceuticals or cosmeceuticals. Such carriers or excipients include, but are not limited to, oils, starch, sucrose and lactose.

Polypeptide. Refers to any chain of amino acids, regardless of length or post-translational modification, for example, glycosylation or phosphorylation.

Polyunsaturated fatty acids (PUFAs). Fatty acids that have at least two double bonds along the carbon backbone. PUFAs can be classified into two major families (depending on the position (n) of the first double bond nearest the methyl end of the fatty acid carbon chain). Thus, the “omega-6 fatty acids” (ω-6 or n-6) have the first unsaturated double bond six carbon atoms from the omega (methyl) end of the molecule and additionally have a total of two or more double bonds, with each subsequent unsaturation occuring 3 additional carbon atoms toward the carboxyl end of the molecule. In contrast, the “omega-3 fatty acids” (ω-3 or n-3) have the first unsaturated double bond three carbon atoms away from the omega end of the molecule and additionally have a total of three or more double bonds, with each subsequent unsaturation occuring 3 additional carbon atoms toward the carboxyl end of the molecule.

Pregnancy food. Refers to a food product formulated for pregnant women.

Prepared food product. Means any pre-packaged food approved for human consumption.

Solid. Refers to the state of matter characterized by resistance to deformation and changes of volume. At the microscopic scale, a solid has these properties: atoms or molecules that comprise the solid are packed closely together, constituent elements that have fixed positions in space relative to each other and if sufficient force is applied, either of these properties can be violated, causing permanent deformation.

### BRIEF DESCRIPTION OF THE DRAWINGS

Exemplary embodiments are illustrated in referenced Figures. It is intended that the embodiments and Figures disclosed herein are to be considered illustrative rather than limiting.

FIG. 1 shows the desaturation and elongation pathways for the n-3 and n-6 PUFAs.

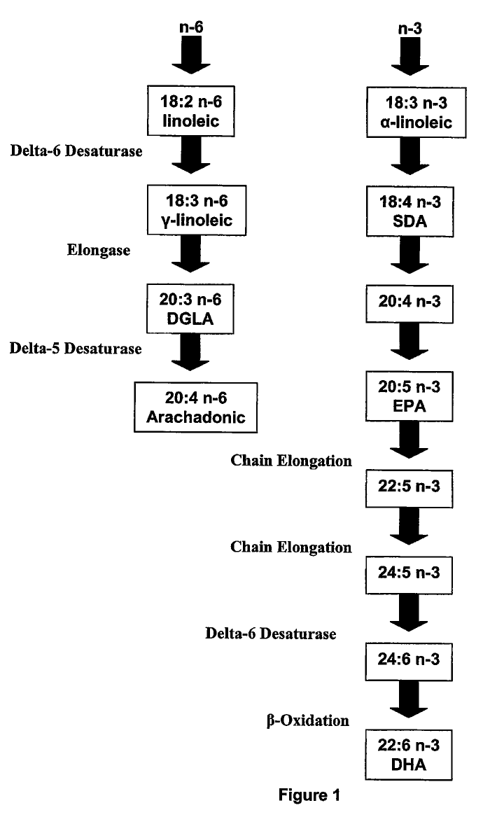

FIG. 2 shows endogenous prostaglandin E₂ concentrations in small intestine from animals consuming Control Diets or Experimental Diets (SDA).

### DETAILED DESCRIPTION OF THE INVENTION

In an especially advantageous embodiment of the present invention, the seeds are of the family Boraginaceae are used; in particular, we have found that the seeds of Buglossoides arvensis are very useful. The present invention is advantageous for the commercial and/or bulk production of an oil with high levels of SDA and low levels of GLA, when compared to others sources, for numerous reasons.

### Current Sources of PUFAs

PUFA-containing oil sources can be found in fish, microbes and plants. Table 1 lists general source types for PUFA-containing oils and their SDA contents. Column 1 shows the source type, column two shows the specific source, column three shows the percentage of SDA in the oil and column four indicates the reference for the data source.

TABLE 1

| | | SDA Content or | |
|---|---|---|---|
| Source Type | Source | Range (%) | Reference |
| Fish Oil | Herring | 1.1-2.8 | 1 |
| | Menhaden | 08.-3.6 | |
| Microbial Oil | Isochrysis galbana | 6.4 | 2 |
| Plant Seed Oil | Blackcurrant | 2.0-4.0 | 3 |
| | (Ribes nigrum) | | |

1. Stansby, M. E., (1981) J. Amer. Oil Chem. Soc., 58: pp. 13
2. Robles Medina, A., et al, (1998) Biotech Adv., 16(3): pp. 517-580
3. Gunstone, F. D., (1992) Prog. Lipid Res., (31): pp. 145-161

### Disadvantages of Current PUFA Sources

### Genetically Modified Organisms

The use of genetically modified organisms, commonly referred to as GMOs, in agriculture has not met with widespread acceptance. For example, genetically engineered canola and soybean lines that produce SDA suffer from a number of short comings, such as developmental cost and lack of commercial popularity with sectors of the general public. Furthermore, GMOs are not allowed for general cultivation in some countries. The use and development of GMOs requires compliance with numerous regulatory agencies that oversee the health of the general public as well as agricultural issues (i.e. the consequences of planting GMO varieties and their impact on the land, water and animals of the cultivated areas). Therefore, there are significant and advantageous reasons from both a commercial and environmental standpoint to use non-GMO plants as PUFA sources whenever possible.

### Marine Organisms

PUFAs obtained from marine oils also have problems. Fish stocks may undergo natural variation and have become significantly depleted by over-fishing. Fish or marine oils have unpleasant tastes and odors, which may not be possible to economically separate from the desired product, and can render such products unacceptable as food supplements. Oily fish and marine oils are known to accumulate undesirable toxins such as heavy metals, pesticide residues and poly-chlorinated biphenyls (PCB's) that are difficult to remove and add to the cost of oil production. Additionally, supplements such as fish oil capsules can contain low levels of the particular desired component and thus require large dosages. High dosages result in ingestion of high levels of undesired components including contaminants. Care must be taken in providing fatty acid supplements as over-addition may result in suppression of endogenous biosynthetic pathways and lead to competition with other necessary fatty acids in various lipid fractions in vivo, leading to undesirable results. Unpleasant tastes and odors of the supplements can make such regimens undesirable, and may inhibit compliance by the patient. Additionally, as the diets of consumers have become increasingly more complex and diverse, PUFAs from marine oils become unsuitable for vegetarians, vegans and various ethnic groups. Furthermore, various ethnic groups exclude any dietary foodstuff consisting of non-plant material from meals, due to religious reasons. Moreover, the isolation of pure omega-3 highly unsaturated fatty acids from this mixture is an involved and expensive process that can result in very high prices.

### Plants as Alternative Sources of PUFAs

There are other natural sources of SDA that have been found in the plant families including the Rosaceaea, Graminaceae, Rosagraceae, Onagraceae, Glossulariaceae, Primulaceae, Saxifragaceae and the Boraginaceae. With the exception of the Boraginaceae and possibly the Primulaceae the above listed plant families are not commercially viable sources of SDA. Within the Boraginaceae family, there are two sub-families of plants that provide the most promising source of SDA to date. These are the Lithospermeae and the Eritrichieae. Buglossoides arvensis, a member of the Lithospermeae sub-family, has been selected for development on the basis of its lipid profile (oil yield, content and fatty acid distribution). The lines of Buglossoides arvensis used have shown surprisingly high seed yield in commercial trials which gives the potential to reduce the cost of production of the desired high SDA oil. Guil-Guerrero, J. L., et al. (2001) “Occurrence and characterization of oils rich in gamma-linolenic acid (III): taxonomical value of the fatty acids in Echium (Boraginaceae)” Phytochemistry 58: 117-120.

Table 2 shows the variability of the total content of oil and the SDA and GLA percentages among various plant species. Column one shows the plant species, column two shows the total percentage of oil obtained from the plant seeds as a percentage of the total weight of the seeds, column three shows the percentage of SDA contained in the oil and column four shows the percentage of GLA contained in the oil.

TABLE 2

| | | % | % |
|---|---|---|---|
| Species | % Total Oil | SDA | GLA |
| Borago officinalis¹ | 30.0 | 0.3 | 23.0 |
| Echium plantagineum¹ | 24.0 | 14.0 | 9.0 |
| Buglossoides arvensis¹ | 20.0 | 20.0 | 5.0 |
| Amsinckia calycina² | 23.2 | 9.9 | 8.8 |
| Lithospermum officinale² | 11.4 | 9.2 | 15.3 |
| Cynoglossum officinale² | 10.7 | 3.1 | 7.9 |
| Cannabis sativa³ | | 0.4 | 1.1 |
| Ribes nigrum⁴ | 6.8 | 2.5 | 16.0 |
| Symphytum officinale⁵ | | 1.2 | 25.8 |

Species ¹ oil quality and yield from internal data from large trial plots or commercial fields
Species ² oil data from Phytochemistry 52 (1999) 423-426 No yield data available
Species ³ oil data from SOFA Database, Aitzetmuller, K. (1996)
Species ⁴ oil data from Ucciani, E., Oleagineu x Corps Gras Lipides Volume 2 (1995) Pp. 491-493
Species ⁵ oil data from Physical and Chemical Characteristics of Oils, Fats and Waxes, J Science Food and Agriculture 54: 309 by Firestone, D.

### Plant Seed as a Source of PUFAs

Echium is currently the only plant species grown commercially for producing SDA-containing oils, however, there are commercial disadvantages to using this plant. For example, relative to Buglossoides, Echium is a low-yielding plant relatively unadapted for agricultural production and hence the derived oil that contains the SDA is costly. Additionally, other plants seeds such as blackcurrant seed, have oil which contains less than 5% SDA. Thus, in order to use these plant materials as a source of SDA, it would be necessary either to use them in large quantities or to carry out expensive chemical processing to concentrate the stearidonic acid. Accordingly, there is a need for a natural and renewable plant material rich in PUFAs that is more commercially and economically advantageous to produce than Echium.

### Economic Comparison of Producing PUFA-Containing Oils Among Plant Species

Table 3 compares seed and oil production and the overall cost of producing a PUFA-containing oil among various plant species. Column one shows the plant species, column two shows the average seed yield in metric tonnes per hectare, column three shows the total percentage of oil obtained from the seed, column four shows the percentage of SDA in the oil, column five shows the percentage of GLA in the oil, column six shows the oil yield in kilograms per hectare, column seven shows the SDA yield in kilograms per hectare, column eight shows the GLA yield in kilograms per hectare, column nine shows the approximate cost of SDA in U.S. dollars per kilogram and column ten shows the approximate cost of the oil in U.S. dollars per metric tonne of oil (prices being subject to current exchange rates). As shown in table 3, Buglossoides has the lowest final cost of SDA production and the lowest overall cost of oil production.

TABLE 3

| | | | | | | | | Approx | |
|---|---|---|---|---|---|---|---|---|---|
| | Seed | % | | | Oil | SDA | GLA | unit cost | Approx |
| | yield | Total | % | % | yield | yield | yield | of SDA | cost of |
| Species | t/ha | oil | SDA | GLA | kg/ha | kg/ha | kg/ha | $/kg | oil $/mt |
| Buglossoides | 0.75 | 20.0 | 20.0 | 5.0 | 150 | 30.0 | 7.5 | 101.0 | 20,200 |
| arvensis | | | | | | | | | |
| Borago | 0.35 | 30.0 | 0.3 | 23.0 | 105 | 0.32 | 24.2 | 784.0 | 23,520 |
| officinalis | | | | | | | | | |
| Echium | 0.25 | 24.0 | 14.0 | 9.0 | 60 | 8.4 | 5.4 | 202.86 | 28,400 |
| plantagineum | | | | | | | | | |
| Amsinckia | <0.1 | 23.2 | 9.9 | 8.8 | 23.2 | 2.3 | 2.0 | 606.1 | >60,000 |
| calycina | | | | | | | | | |
| Lithospermum | <0.1 | 11.4 | 9.2 | 15.3 | 11.4 | 1.0 | 1.7 | 526.3 | >60,000 |
| officinale | | | | | | | | | |
| Cynoglossum | <0.1 | 10.7 | 3.1 | 7.9 | 10.7 | 0.33 | 0.8 | 560.7 | >60,000 |
| officinale | | | | | | | | | |

### Health Benefits of Consuming PUFAs

PUFA oil dietary supplementation is known to have beneficial health effects. Glycogen storage disease is an inherited disorder, and is often complicated by severe hyperlipoproteinemia and hypercholesterolemia, which increases the risk of premature atherosclerosis and cardiovascular diseases. It has been reported that patients suffering from glycogen storage disease that received 10 grams of fish oil for 3 months experienced a significant decrease in levels of triglycerides in the blood serum (−49%) and cholesterol levels in the blood serum (−23%), and a reduction in LDL levels in the blood serum (−40%), and a significant increase in HDL levels in the blood serum (+30%) (Levy, E., et al., I. Am. J. Clin. Nutr., 57:922-29 (1993)).

Omega-3 highly unsaturated fatty acids are of significant commercial interest in that they have been recently recognized as important dietary compounds for preventing arteriosclerosis and coronary heart disease, for alleviating inflammatory conditions and for retarding the growth of tumor cells. These beneficial effects are a result both of omega-3 highly unsaturated fatty acids causing competitive inhibition of compounds produced from omega-6 fatty acids, and from beneficial compounds produced directly from the omega-3 highly unsaturated fatty acids themselves (Simopoulos et al., 1986). Omega-6 fatty acids are the predominant highly unsaturated fatty acids found in plants and animals. Currently the only commercially available dietary source of omega-3 highly unsaturated fatty acids is from certain fish oils which can contain up to 20-30% of these fatty acids.

### FURTHER EMBODIMENTS OF THE INVENTION

An important advantage of the present invention is that the oil of the present invention contains both SDA in an amount higher than conventional oil producing plant seeds and GLA in an amount lower than conventional oil producing plant seeds.

The present invention encompasses an oil derived from Buglossoides arvensis having and SDA content, measured as a percentage by weight, ranging unexpectedly between any of the following numbers: 14.0%, 14.1%, 14.5%, 14.7%, 15.0%, 15.3%, 15.6%, 16.0%, 16.2%, 16.6%, 16.9%, 17.0%, 17.3%, 17.5%, 17.9% or higher, and including all fractions thereof and a GLA content, measured as a percentage by weight, ranging unexpectedly between any of the following numbers 7.0%, 6.8%, 6.5%, 6.2%, 5.9%, 5.7%, 5.4%, 5.0%, 4.8%, 4.6%, 4.4%, 4.0%, 3.5%, 3.3%, 3.0%, 2.9%, 2.5%, 2.3%, 2.0%, 1.9%, 1.7%, 1.4%, 1.2%, 0.8%, 0.6%, 0.4% and 0.3% or lower, and including all fractions thereof.

The present invention also encompasses an oil from Bugloissoides arvensis with a preferred ratio of SDA:GLA of about between 3:1 to about 4:1.

The present invention also encompasses an oil from Buglossoides arvensis that can be obtained from the seeds of Buglossoides arvensis without any further unit operations to increase the concentration of SDA in the oil itself.

The present invention also encompasses a composition containing the oil of the present invention which may also be added to food even when supplementation of the diet is not required. For example, the composition may be added to food of any type including but not limited to margarines, modified butters, cheeses, milk, yogurt, chocolate, candy, snacks, salad oils, cooking oils, cooking fats, meats, fish and beverages.

The oil of the present invention can also be used in a wide variety of topical applications, including many cosmetic and dermatological applications.

The oil of the present invention by itself, or in compositions formed using the oil itself, can be used to treat a wide variety of skin disorders, including but not limited to the following, such as dry skin, itchy skin, psoriasis, eczema and the like. For example, the oil extract can be used in topical compositions including skin creams, lotions, serums and emulsions, including cleansers, moisturizing creams, sunscreens, shampoos and bath oils.

The present invention also encompasses a composition for the topical application by the human or animal body, comprising an oil extracted from the seeds of Buglossoides arvensis, or in combination with a physiologically acceptable carrier. Where such carriers are determined to be necessary, for example to alter or change the physical characteristics of the product, the precise nature of the carrier would depend on the use desired for the composition. In addition, the carrier would usually contain other active ingredients, such as moisturizers (e.g. for moisturizing cream), surfactants (e.g. for shampoo) or a UV-blocking/absorbing compound (e.g. for a sun-cream). Some specific examples of suitable carriers are disclosed below.

The carrier can, optionally, include materials normally present in personal care or healthcare formulations, such as antiseptic compounds, emollients, inorganics, humectants, moisturizers, anti-inflammatory agents, vitamins, preservatives, pH adjusters, proteins, herbal extracts, carriers/solvents, soothing/cooling agents, antioxidants, perfumes, emulsifiers and viscosity modifiers and other lipids such as triglycerides, phospholipids and sphingolipids. Specific examples of useful materials include glycerine, sodium pyrrolidone carboxylate, triethanolamine stearate, sorbitan esters, alkoxylated fatty acids, alkoxylated fatty alcohols, alkoxylated mono/di-glycerides, vitamin E, lanolin, lanolin alcohols, lanolin esters, cholesterol, cholesterol esters, phytosterols, titanium dioxide, zinc oxide, allantoin, calamine, sodium lactate, water, lactic acid, pro-vitamin B₅ and menthol. Optional acceptable antioxidants include, but are not restricted by, hindered phenolics antioxidants such as butylated hydroxy toluene, butylated hydroxy anisole, butylated hydroquinone; gallate esters, ascorbic acid, ascorbic acid esters, plant extracts such as rosemary oil, rosmarinic acid and combinations of suitable antioxidants.

The oil of the present invention can be delivered orally in a hard or soft gel capsule, where the capsule contains an amount of the oil ranging between any of the following numbers: 0.01 grams, 0.05 grams, 0.08 grams, 0.10 grams, 0.12 grams, 0.15 grams, 0.17 grams, 0.18 grams, 0.19 grams, 0.25 grams, 0.29 grams, 0.33 grams, 0.38 grams, 0.42 grams, 0.50 grams, 0.53 grams, 0.59 grams, 0.61 grams, 0.66 grams, 0.69 grams, 0.75 grams, 0.78 grams, 0.83 grams, 0.85 grams, 0.87 grams, 0.91 grams, 0.96 grams, 1.01 grams, 1.08 grams, 1.12 grams, 1.16 grams, 1.22 grams, 1.32 grams, 1.39 grams, 1.43 grams, 1.45 grams, 1.49 grams, 1.53 grams, 1.58 grams, 1.62 grams, 1.64 grams, 1.70 grams, 1.75 grams, 1.79 grams, 1.83 grams, 1.88 grams, 1.91 grams, 1.94 grams, 1.99 grams and 2.0 grams, and including all fractions thereof.

The present invention also encompasses a soft gel or hard capsule containing a composition, wherein the oil of the present invention comprises a percentage of the total composition in the soft gel or hard capsule ranging between any of the following numbers: 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25.0%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 41.5%, 42.0%, 42.5%, 43.0%, 43.5%, 44.0%, 44.5%, 45.0%, 45.5%, 46.0%, 46.5%, 47.0%, 47.5%, 48.0%, 48.5%, 49.0%, 49.5%, 50.0%, 50.5%, 51.5%, 52.0%, 52.5%, 53.0%, 53.5%, 54.0%, 54.5%, 55.0%, 55.5%, 56.0%, 56.5%, 57.0%, 57.5%, 58.0%, 58.5%, 59.0%, 59.5%, 60.0%, 60.5%, 61.0%, 61.5%, 62.0%, 62.5%, 63.0%, 63.5%, 64.0%, 64.5%, 65.0%, 65.5%, 66.0%, 66.5%, 67.0%, 67.5%, 68.0%, 68.5%, 69.0%, 69.5%, 70.0%, 70.5%, 71.0%, 71.5%, 72.0%, 72.5%, 73.0%, 73.5%, 74.0%, 74.5%, 75.0%, 75.5%, 76.0%, 76.5%, 77.0%, 77.5%, 78.0%, 78.5%, 79.0%, 79.5%, 80.0%, 80.5%, 81.0%, 81.5%, 82.0%, 82.5%, 83.0%, 83.5%, 84.0%, 84.5%, 85.0%, 85.5%, 86.0%, 86.5%, 87.0%, 87.5%, 88.0%, 88.5%, 89.0%, 89.5%, 90.0%, 90.5%, 91.0%, 91.5%, 92.0%, 92.5%, 93.0%, 93.5%, 94.0%, 94.5%, 95.0%, 95.5%, 96.0%, 96.5%, 97.0%, 97.5%, 98.0%, 98.5%, 99.0%, 99.5% and 100.0%, and including all fractions thereof.

Suitable compositions for pharmaceutical presentation are discussed in detail, for example, in Williams British Patent Specification No. 1,082,624, to which reference may be made, and are in any case very well known generally for any particular kind of preparation. Also see U.S. Pat. No. 5,178,873 by Horrobin, et al. Thus, for example, tablets, hard or soft gelatin or other capsules, enteric-coated capsules, ingestible liquid or powder preparations can be prepared as required, and topical preparations when the acids are to be absorbed through the skin or by other direct application. Injectable solutions may be prepared in various ways including the use of free albumin to solubilise free acids, or the preparation of lipid emulsions, liposomes or the use of water soluble salts such as the lithium or sodium or meglumine salts.

Advantageously, an antioxidant and/or stabilizer is incorporated into the composition of the present invention. Alpha-tocopherol in concentration of about 0.1% by weight has been found suitable to be a preservative and is one of a number of possible stabilizers well known in the field. In addition, natural or synthetic vitamin E, tocopherols, hindered phenolics such as butylated hydroxy anisole (BHA), di-tert-butylhydroxytoluene (BHT), tert-butylhydroquinone (TBHQ), gallate esters such as propyl gallate and octyl gallate, vitamin C derivatives such as ascorbyl palmitate, ascorbyl stearate, plant extracts such as rosemary extract, green tea extract or any combination of the above, may be added.

It will be understood that the absolute quantity of active materials present in any dosage unit should not exceed that appropriate to the rate and manner of administration to be employed but on the other hand should also desirably be adequate to allow the desired rate of administration to be achieved by a small number of doses. The rate of administration will moreover depend on the precise pharmacological action desired.

The oil of the present invention can also be coated onto small particles, wherein the particles optimally retain the stability and handling characteristics of solid flow-able powders. Further, liquids processed into solid powder form have been found to be less susceptible to deterioration through excessive temperature, volatilization or reaction with oxygen. The particles that are coated may be carrier particles or active particles. Active particles generally are those that will be part of the desired material to be delivered. Carrier particles generally are those that are relatively inert in the sense that they are not part of the desired material to be delivered. See WO 2004/016720.

Unfortunately, PUFAs are very susceptible to oxidation due to the high degree of unsaturation. When oxidized, the fatty acids turn rancid producing an unpleasant smell and taste. This means that for PUFAs to be incorporated into food components, they have to be protected against oxidation. The coating of these materials affords protection to these ingredients and allows them to be delivered at the target site at the required time. Shelf-life and stability of PUFAs is improved due to the inhibition of oxidation. Other benefits of coating include the ease of handling of the material (s) due to the small particulate, powder form of the coated PUFA-containing material or coated PUFA matrix particle and the suitability for incorporation at any of a variety of stages of preparation of many differing types of foodstuffs and nutritional compositions.

Microencapsulation has been defined as a process by which small particles (generally between 1 to 1000 microns in diameter) of solid, liquid or gas are packaged within a secondary material to form a microcapsule. (Sanguansri et. al., Microencapsulation for Innovative Ingredients a Scoping Study: opportunities for Research into the Microencapsulation of Food Ingredients, Food Science Australia, May 2001). Vasishtha, PreparedFoods,“Microencapsulation: Delivering a market advantage (July 2002) provides an overview of the types of core and coating materials and encapsulation techniques used in the industry.

A variety of encapsulation processes are mentioned and include both physical and chemical techniques. Examples of physical techniques mentioned are spray-drying, the spinning disc and co-extrusion processes. Examples of chemical techniques mentioned are phase separation, gelation and coacervation.

U.S. Pat. No. 6,048,557, issued to Van Den Berg et al. on Apr. 11, 2002, describes PUFA encapsulated solid carrier particle for use as a foodstuff. Specifically, solid carrier particles are provided on to which have been encapsulated, or absorbed, at least one PUFA in a liquid form.

WO 2001/74175, published Oct. 11, 2001, described encapsulation of food ingredients, in particular, oxygen sensitive oils or oil soluble ingredients.

U.S. Pat. No. 4,895,725, issued to Kantor et. al. on Jan. 23, 1990, describes microencapsulation of fish oils which are formed by preparing an emulsion of an oil-based biologically active compound and a non-oil soluble enteric coating in a basic solution, atomizing the emulsion into an acidic aqueous solution, and separating the precipitated microcapsules from the acidic aqueous solution.

U.S. Pat. No. 6,234,464B1, issued to Krumbholz et. al. on May 22, 2001, describes microencapsulated unsaturated fatty acids or fatty acid compounds or mixtures thereof involving two layers. The inner layer is composed of gelatin A, gelatin B, casein or an alginate or of a derivative or salt of one of these polymers. The outer layer is composed of gelatin B, gum arabic, pectin or chitosan or a derivative or salt of one of these polymers.

U.S. Pat. No. 4,217,370, issued to Rawlings et. al. on Aug. 12, 1980, describes lipid-containing feed supplements and foodstuffs made by admixing a lipid material to form an emulsion and adjusting the pH to lower it to its isoelectric point, thereby aggregating the protein and simultaneously microencapsulating the lipid.

The formulation of the carrier also depends on the form required for the composition. Broadly, the composition may be in the form of a solid or a liquid suitable for topical application (aerosol). Typically, the pharmaceutical composition is provided in the form of an oil, lotion, cream, an emulsion, dispersion or a gel; however, the pharmaceutical composition may be provided in other forms, such as a capsule, tablet, injectable liquid, suppository or solid stick.

The amount of the SDA and GLA in the pharmaceutical composition depends upon the way the composition is to be used. However, the compositions according to the invention typically contain 1 percent by weight to 17 percent by weight of SDA and less than 6 percent by weight of GLA.

According to another aspect of the invention there is provided a composition for oral ingestion to the human or animal body, comprising an oil extracted from the seeds of Buglossoides arvensis, optionally in combination with a physiologically acceptable carrier.

The oil also has a wide range of nutritional uses. For example, the oil can be provided as an additive to existing food products, for instance as an additive to fruit juice, milk, milk-based drinks, probiotic drinks, yoghurt, vegetable spreads, butter; it may be in the form of a nutritional supplement (e.g. a vitamin-containing supplement), and may be provided in solid form, for example as a tablet, as a soft or hard gelatin capsule, or in liquid form.

The amount of oil in the composition also depends upon the desired use. However, for most applications, an amount of the oil in the range of from 1 percent by weight to 100 percent by weight is sufficient. Providing between 0.2 percent by weight and 18 percent by weight of SDA and from 0.05 percent by weight to 5.5 percent by weight GLA is an appropriate amount of the oil in the composition.

The compositions according to the invention may be provided in a bottle, a tube or any other suitable packaging. The container for the compositions may be provided with dispensing means for dispensing the composition. Any known form of dispensing means may be used. When the composition is a liquid, it may be desirable to employ a dispensing means that can dispense it in the form of a spray.

We have described above the use of oil extracted from the seeds of Buglossoides arvensis, and compositions including such oils. This oil contains high levels of SDA and low levels of GLA.

The present invention also includes the use of SDA and GLA formed from an oil extracted from seeds of Buglossoides arvensis in topical application to or for oral ingestion by, the human or animal body. The SDA and GLA so formed may be used alone for these purposes or, preferably, it is used to form part of a composition for topical application to or oral ingestion by, the human or animal body. The SDA may be part of a mixture of essential fatty acids formed from an oil extracted from the seed of Buglossoides arvensis.

The examples which follow are intended to illustrate certain preferred embodiments of the invention and no limitation of the invention is implied.

### EXAMPLES

### Example 1

### Extraction of Oil from Plant Seeds

Methods used to extract oil from seeds are well known to those skilled in the art and include crushing seed to expel the oil and or extracting the oil from the crushed seed with suitable solvents including organic solvents such as hexane, esters and alcohols, inorganic solvents such as water and supercritical fluids such as supercritical carbon dioxide. The oil extract can be optionally treated to remove impurities by filtration, washing, alkali refining, bleaching, deodorization, de-gumming and treatment with absorbants such as activated charcoal, alumina, montmorrilonite clays, molecular sieves and the like. The oil can be optionally treated with stabilizers and antioxidants to improve shelf-life and appearance. Oil processing is described in detail in “The Lipids Handbook” edited by Frank D. Gunstone, John L. Harwood and Fred D. Padley, published (1986) Chapman and Hall Ltd., ISBN 0 412 24480 2, pages 181-215.

### Example 2

### Purification of PUFAs from Oil

In general, means for the purification of PUFAs include extraction with organic solvents, sonication, supercritical fluid extraction (e.g., using carbon dioxide), saponification and physical means such as presses or combinations thereof. Of particular interest is extraction with methanol and chloroform in the presence of water. E. G. Bligh & W. J. Dyer. (1959) Can. J. Biochem. Physiol. 37:911 917. Where desirable, the aqueous layer is acidified to protonate negatively-charged moieties and thereby increase partitioning of desired products into the organic layer. After extraction, the organic solvents are removed by evaporation under a stream of nitrogen. When isolated in conjugated forms, the products are enzymatically or chemically cleaved to release the free fatty acid or a less complex conjugate of interest and are subject to further manipulations to produce a desired end product. Desirably, conjugated forms of fatty acids are cleaved with potassium hydroxide.

If further purification is necessary, standard methods are employed. Such methods may include extraction, treatment with urea, fractional crystallization, HPLC, fractional distillation, silica gel chromatography, high-speed centrifugation or distillation, or combinations of these techniques. Protection of reactive groups, such as the acid or alkenyl groups, are performed at any step through known techniques (e.g., alkylation, iodination). Methods used include methylation of the fatty acids to produce methyl esters. Similarly, protecting groups are removed at any step. Desirably, purification of fractions containing GLA, SDA, STA, ARA, DHA and EPA is accomplished by treatment with urea and/or fractional distillation.

### Example 3

### Comparison of the Fatty Acid Composition of Buglosssoides arvensis with Echium plantageneum

In Table 4, the results of trials growing Buglossoides arvensis and Echium plantageneum under rigorously controlled conditions of sowing rate, nutrient input and harvesting methods. Fatty acid analysis and seed oil content for replicated trials of these plant species are shown in columns A and B (Buglossoides arvensis) and column C (Echium plantageneum). Unexpectedly, the gamma linolenic acid percentages were lower for Buglossoides arvensis when compared to Echium plantageneum and the stearidonic acid percentages were higher for Buglossoides arvensis than Echium plantageneum, even though the overall oil content percentages are similar.

TABLE 4

| | | Reference Trials | | |
|---|---|---|---|---|
| Molecular | | (gc area, %) | | |
| Abbreviation | Fatty acid | A | B | C |
| C14:0 | myristic acid | | | |
| C16:0 | palmitic acid | 4.27 | 4.54 | 7.01 |
| C18:0 | stearic acid | 1.81 | 1.99 | 3.61 |
| C18:n-9 | oleic acid | 7.01 | 7.39 | 16.41 |
| C18:1n-11 | vaccenic acid | 0.59 | 0.61 | |
| C18:2n-6 | linoleic acid | 11.17 | 12.00 | 14.96 |
| C18:3n-6 | γ-linolenic acid | 5.32 | 5.29 | 11.83 |
| C18:6n-3 | α-linolenic acid | 38.83 | 41.15 | 28.98 |
| C18:4n-3 | stearidonic | 17.99 | 17.27 | 12.99 |
| | acid | | | |
| C20:0 | arachidic acid | | | 0.39 |
| C20:1n-9 | gadoleic acid | 0.84 | 0.75 | 0.68 |
| C22:0 | behenic acid | | | |
| C22:1n-9 | erucic acid | | | 0.13 |
| C24:0 | lignoceric acid | | | |
| C24:1n-9 | nervonic acid | | | 0.14 |

| | | | |
|---|---|---|---|
| Oil content (%) | 22.2 | 21.9 | 21.0 |

### Example 4

### Comparison of Yield Characteristics Between Buglossoides and Echium

Table 5 compares yield data for field production of Echium and Buglossoides. Row 1 shows the seed yield in kilograms per hectare, row two shows the oil content of the seed as a percentage, row three shows the oil yield in kilograms per hectare and row four shows the SDA yield in kilograms per hectare. The results indicate that the seed yield of Buglossoides is unexpectedly 3 times greater than Echium, the oil yield in (kg/ha) of Buglossoides is unexpectedly 2.5 times greater than Echium and the SDA yield of Buglossoides is unexpectedly 3.5 times greater than Echium.

TABLE 5

| Yield Characteristic | Echium | Buglossoides |
|---|---|---|
| Seed yield (kg/ha) | 250 | 750 |
| Oil content (%) | 24 | 20 |
| Oil yield (kg/ha) | 60 | 150 |
| SDA yield (kg/ha) | 8.4 | 30 |

### Example 5

### 2004 Yield data of Buglossoides arvensis

Table 6 compares the fatty acid profiles of B. arvensis designated as Line 1 with B. arvensis plants grown from seed whose source is the Royal Botanical Gardens, Kew, United Kingdom. Both the Kew and Line 1 were grown in direct side by side comparison. The Sites listed in row two designate various locations in the United Kingdom. Seed Rate/Yield data was determined from values obtained at Site 4. No other yield assessments were carried out at the other Sites. The average oil content of the seed unexpectedly averaged 20.16% for Line 1 and 22.2% for the Kew stock.

TABLE 6

| | | | Oil quality | | | | |
|---|---|---|---|---|---|---|---|
| | | Line 1 | Line 1/ | Line 1/ | Line 1/ | Line 1/ | |
| Fatty Acid | Kew Stock | Stock | Site 1 | Site 2 | Site 3 | Site 4 | Average |
| C16:0 palmitic acid | 4.27 | 4.54 | | | | | |
| C18:0 stearic acid | 1.81 | 1.99 | | | | | |
| C18:1 w9 oleic acid | 7.01 | 7.39 | | | | | |
| C18:1 w7 vaccenic | 0.59 | 0.61 | | | | | |
| acid | | | | | | | |
| C18:2 linoleic acid | 11.17 | 12.00 | | | | | |
| C18:3 GLA | 5.32 | 5.29 | 5.73 | 5.83 | 6.05 | 5.91 | 5.69 |
| C18:3 ALA | 38.83 | 41.15 | 43.67 | 44.93 | 44.81 | | 42.68 |
| C18:4 stearidonic | 17.99 | 17.27 | 18.28 | 19.59 | 19.44 | 18.35 | 18.49 |
| acid | | | | | | | |
| C20:1 eicosenoic | 0.84 | 0.75 | | | | | |
| acid | | | | | | | |
| % of total oil | 88.29 | 91.22 | | | | | |
| Oil content of seed | 22.2 | | 20.04 | 18.58 | 19.69 | 18.56 | 20.16 |

### Example 6

### 2006 Yield Data of Buglossoides arvensis

Table 7 compares details the fatty acid profile of seeds harvested from a 0.4 hectare plot located in Cambridgeshire, United Kingdom in August 2006. The seed yield of this plot was calculated to be approximately 700 kg/ha.

TABLE 7

| | Percentage of total |
|---|---|
| Fatty acid | fatty acid |
| C14:0 | |
| C16:0 | 5.66 |
| C16:1 | 0.18 |
| C16:2 | 0.07 |
| C18:0 | 2.08 |
| C18:1 | 9.62 |
| C18:2 | 13.55 |
| C18:3 gamma | 6.47 |
| C18:3 | 42.00 |
| C18:4 | 19.11 |
| C20:0 | |
| C20:1 | 0.80 |
| C20:2 | 0.09 |
| C20:3 | |
| C22:0 | 0.10 |
| C22:1 | 0.14 |
| C24:0 | |
| C24:1 | 0.12 |
| Total | 99.99 |

### Example 7

### Use of the Present Invention in Diets for Mice

### Protocol for Preparing Experimental and Control Diet Groups of Mice

BALB/c mice (19-21 g) were separated into 2 experimental groups of 7 animals per group. The Control Diet group of animals consumed a modification of the MONSANTO US17 Rodent Diet supplemented with 0.1 g arachidonate ethyl ester/Kg of diet (Research Diets) that was designed to represent the typical North American rodent diet with respect to energy distribution and fatty acid content. Petrik M. B., et al., (2000) Highly unsaturated (n-3) fatty acids, but not alpha-linolenic, conjugated linoleic or gamma-linolenic acids, reduce tumorigenesis in Apc(Min/+) mice. J Nutr. 130:2434-43. The Experimental Diet group, or the SDA treatment group of the present invention, consumed a diet with 1% of energy supplied as SDA (the human equivalent of approximately 2.2 g SDA/day). The total energy from fat was equivalent in both groups with the SDA largely replacing oleic acid in the Experimental Diet.

Table 8 shows the energy distribution of the Control group diet and the Experimental group. Column one shows the main nutritional components, columns two and three show that the breakdown of the kilocalorie percentages for each nutritional component of the Control Diet and the Experimental Diet, respectively, are identical but vary in the fatty acid composition, as shown in Table 9.

TABLE 8

| | |
|---|---|
| Control Diet | Experimental Diet |

| | |
|---|---|
| Component | Percent kilocalories |

| | | |
|---|---|---|
| Protein | 16.0 | 16.0 |
| Carbohydrate | 50.3 | 50.3 |
| Fat | 33.7 | 33.7 |

Table 9 shows the fatty acid composition of the Control Diet group and the Experimental diet group enriched in 18:4 n-3. The Experimental Diet group contained 2.6% w/w of Buglossoides oil, compared to the Control Diet group which contained no Buglossoides oil. Column one shows the fatty acid and columns two and three show the percentage of each fatty acid for the Control Diet group and the Experimental Diet group, respectively.

TABLE 9

| | |
|---|---|
| Control Diet | Experimental Diet |

| | |
|---|---|
| Fatty Acid | Percent total fatty acids |

| | | |
|---|---|---|
| C14:0 | 0.6 | 0.5 |
| C16:0 | 24.5 | 24.6 |
| C16:1 | 0.2 | 0.2 |
| C18:0 | 11.7 | 11.6 |
| C18:1 n-9 | 39.6 | 35.4 |
| C18:1 n-7 | 0.9 | 0.8 |
| C18:2 | 17.3 | 16.3 |
| C18:3 n-6 | 0.0 | 0.6 |
| C18:3 n-3 | 5.1 | 5.9 |
| C18:4 n-3 | 0.0 | 3.0 |
| C20:4 n-6 | 0.1 | 0.1 |

Table 10 shows the specific diet composition of the Control Diet group and the Experimental Diet group. Column one shows the fatty acid component and columns two and three show the number of grams for each component for the Control Diet group and the Experimental Diet group, respectively. The addition of Buglossoides oil to the Experimental Diet group was compensated by the removal of linseed oil and some of the safflower and sunflower oils as shown in Table 10. This allowed both the Experimental Diet group and the Control Diet groups to maintain the same fat content (33.7% of the total calories as show in Table 8). Since the total fat content of the two diets was maintained, the added 18:3 n-6 and 18:4 n-3 provided by Buglossoides oil had to replace another fatty acid component, where the dietary oil replacement targeted oleic acid, 18:1 n-9, as the fatty acid to be reduced in the Experimental Diet group (see Table 9).

TABLE 10

| | |
|---|---|
| Control Diet | Experimental Diet |

| | |
|---|---|
| Component | Grams |

| | | |
|---|---|---|
| Cocoa Butter, Deodorized | 35.3 | 35.3 |
| Linseed Oil, RBD | 12.6 | 0 |
| Palm Oil, Bleached, | 49.4 | 49.4 |
| Deodorized | | |
| Safflower Oil, USP | 21.8 | 18.8 |
| Sunflower Oil, Trisun Extra | 23.7 | 16.8 |
| Arachidonic Acid, Ethyl | 0.1 | 0.1 |
| Ester | | |
| Eicosapentaenoic Acid, | 0 | 0 |
| Ethyl Ester | | |
| Docosahexaenoic Acid, | 0 | 0 |
| Ethyl Ester | | |
| Oleic Acid, Ethyl Ester | 0 | 0 |
| Buglossoides Oil | 0 | 22.5 |
| Non-lipid constituents | 730 | 730 |

Each group (both the Experimental Diet and Control Diet groups) consumed their respective diet for 3 weeks. Mice from each group were then sacrificed by guillotine and their tissues were immediately removed and processed for analyses.

Analyses of Mice Tissues from Both the Experimental and Control Groups of Mice

Liver or small intestine homogenization was performed in ice-cold phosphate-buffered saline. For intestinal tissue, the homogenization buffer also contained the cyclooxygenase inhibitor indomethacin (1 mM) to prevent the production of prostaglandins during sample preparation. Id. Lipids were extracted from tissue homogenates. Bligh E. G., et al., (1959) A rapid method of total lipid extraction and purification. Can. J. Biochem. Physiol. 37:911-17. Fatty acyl methyl esters were then prepared and measured by gas chromatography with flame ionization detection. Surette, M. E., et al., (2003) Inhibition of leukotriene synthesis, pharmacokinetics, and tolerability of a novel dietary fatty acid formulation in healthy adult subjects. Clin. Ther. 25:948-71.

Endogenous prostaglandin E₂ content in the small intestine homogenates was measured by ELISA (Cayman Chemical Co). Protein content was measured by a modification of the Lowry method.

Table 11 compares the fatty acid composition of the liver tissues in mice between the Control Diet group and the Experimental Diet group enriched in 18:4 n-3. Column one shows the fatty acids and columns two and three show the fatty acid composition, as a percentage of the total fatty acid in the liver tissue in the Control Diet group and the Experimental Diet group, respectively.

TABLE 11

| | |
|---|---|
| Control Diet | Experimental Diet |

| | |
|---|---|
| Fatty Acid | (Percent of total) |

| | | |
|---|---|---|
| 16:0 | 25.0 ± 0.4 | 26.1 ± 0.2 |
| 16:1 | 0.9 ± 0.1 | 0.9 ± 0.1 |
| 18:0 | 10.7 ± 0.5 | 12.3 ± 0.6 |
| 18:1 n-9 | 30.7 ± 0.7 | 24.5 ± 1.5* |
| 18:1 n-7 | 1.3 ± 0.1 | 1.2 ± 0.1 |
| 18:2 | 14.2 ± 0.1 | 13.6 ± 0.3 |
| 18:3 n-6 | ND | ND |
| 18:3 n-3 | 0.9 ± 0.1 | 1.2 ± 0.1 |
| 18:4 n-3 | 0.05 ± 0.01 | 0.2 ± 0.02* |
| 20:1 n-9 | 0.5 ± 0.01 | 0.4 ± 0.03 |
| 20:2 n-9 | 0.4 ± 0.02 | 0.3 ± 0.02 |
| 20:3 n-6 | 0.9 ± 0.04 | 1.4 ± 0.1* |
| 20:4 n-6 | 9.0 ± 0.6 | 9.7 ± 0.8 |
| 20:5 n-3 | 0.4 ± 0.02 | 1.7 ± 0.1* |
| 22:4 n-6 | 0.21 ± 0.01 | 0.16 ± 0.01* |
| 22:5 n-3 | 0.2 ± 0.01 | 0.5 ± 0.03* |
| 22:6 n-3 | 4.4 ± 0.2 | 5.8 ± 0.2* |

Values represent means ± SEM.
ND = Mean value less than 0.05% of total fatty acids.
*Significantly different from Control diet group as determined by two-tailed t-test, p < 0.05

Table 12 compares the fatty acid composition of the small intestine in mice between the Control Diet group and the Experimental Diet group. Column one shows the fatty acids and columns two and three show the fatty acid composition, as a percentage of the total fatty acid in the small intestine in the Control Diet group and the Experimental Diet group, respectively.

TABLE 12

| | |
|---|---|
| Control Diet | Experimental Diet |

| | |
|---|---|
| Fatty Acid | (Percent of total) |

| | | |
|---|---|---|
| 16:0 | 16.5 ± 0.5 | 15.8 ± 0.2 |
| 16:1 | 0.5 ± 0.02 | 0.4 ± 0.04 |
| 18:0 | 25.0 ± 0.4 | 26.7 ± 0.2 |
| 18:1 n-9 | 14.8 ± 0.6 | 12.9 ± 0.7 |
| 18:1 n-7 | 1.6 ± 0.1 | 1.4 ± 0.03 |
| 18:2 | 15.7 ± 0.7 | 16.0 ± 0.1 |
| 18:3 n-6 | ND | ND |
| 18:3 n-3 | 0.4 ± 0.1 | 0.5 ± 0.04 |
| 18:4 n-3 | ND | ND |
| 20:3 n-6 | 2.5 ± 0.1 | 3.1 ± 0.1* |
| 20:4 n-6 | 15.9 ± 0.5 | 14.5 ± 0.7 |
| 20:5 n-3 | 0.7 ± 0.1 | 2.1 ± 0.1* |
| 22:4 n-6 | 1.5 ± 0.2 | 1.1 ± 0.02* |
| 22:5 n-3 | 0.5 ± 0.03 | 0.8 ± 0.02* |
| 22:6 n-3 | 4.3 ± 0.1 | 4.5 ± 0.1 |

Values represent means ± SEM.
ND = Mean value less than less than 0.05% of total fatty acids.
*Significantly different from control as determined by two-tailed t-test, p < 0.05.

Summary of Data from Mice Tissue Analyses Between the Control Diet Group and the Experimental Diet Group

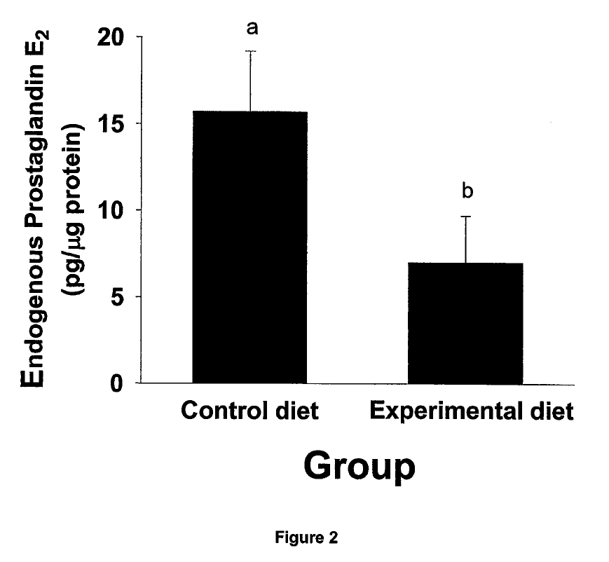

The results of tissue fatty acid analyses show that the consumption of the Experimental Diet containing Buglossoides oil resulted in an unexpected increase in the tissue content of long chain n-3 fatty acids compared to animals consuming the Control Diet. Specifically, the eicosapentaenoic acid (20:5 n-3) and docosapentaenoic acid (22:5 n-3) content in both liver and intestine were unexpectedly increased in animals consuming the Experimental Diet. Further, in the liver tissue, the content of docosahexaenoic acid was also unexpectedly increased indicating that the dietary 18:4 n-3 was metabolized to 22:6 n-3. Both tissues showed an increase in dihomogammalinolenic acid (20:3 n-6) content in animals consuming the Experimental Diet, which was likely due to increased consumption of 18:3 n-6 in the Experimental Diet. These changes in tissue fatty acid composition indicate an unexpected anti-inflammatory effect of the Experimental Diet. When endogenous prostaglandin E₂ was measured in intestines from animals consuming the Experimental Diet, there was significantly less prostaglandin E₂ content than in the small intestines from animals consuming the Control Diets, unexpectedly confirming the anti-inflammatory properties of the Experimental Diet (See FIG. 2 showing endogenous prostaglandin E₂ concentrations in small intestine from animals consuming Control Diets or Experimental Diets (SDA). Values represent means±SD. Values with no common superscript are significantly different as determined by two-tailed student's T-test, p<0.05).

### Example 8

### Production of Cosmeceutical Products Containing the Present Invention

A preferred composition of the present invention is for use in cosmeceutical compositions, whether topical or oral. Such cosmeceutical compositions include, but are not limited to, topical oils (e.g., sunscreen oil, facial oils, bath oil), topical creams (e.g., face cream, sunscreen lotion) and oral compositions (e.g., capsules) which contains the oil of the present invention.

Cosmeceutical compositions within the scope of the invention were prepared. A base formulation in Table 13 was made by heating the oil phase and the water phase separately to 65° C. to 70° C. The water phase was added to the oil phase with stirring. The composition was stirred to cool and perfume was added at 40° C.

TABLE 13
Formula for Protective Moisturizing Lotion

| Component | Percentage by Weight |
|---|---|
| Isopropyl Myristate | 5.0 |
| Cetyl Phosphate | 3.0 |
| Glyceryl Stearate | 2.0 |
| Buglossoides arvensis | 2.0 |
| Cetyl Alcohol | 2.0 |
| BHT | 0.05 |
| Deionized Water | to 100 |
| Urea | 5.0 |
| Propylene Glycol | 3.0 |
| Potassium Hydroxide (50%) | 1.0 |
| EDTA | 0.1 |
| Perfume, Preservative, Color | qs |

Another cosmeceutical composition within the scope of the invention was prepared. A base formulation in Table 14 was prepared by heating the oil phase and water phase separately to 65° C. to 70° C. The water phase was added to the oil phase with stirring. The pH was adjusted to 7.0 to 7.5 with potassium hydroxide and was stirred to cool. Perfume was added at 40° C. to 45° C.

TABLE 14
After-sun Lotion with Anti-inflammatory PUFAs

| Component | Percentage by Weight |
|---|---|
| Paraffinum Liquidum | 5.0 |
| Stearic Acid | 3.0 |
| Buglossoides arvensis | 3.0 |
| Glyceryl Stearate and PEG-100 Stearate | 2.0 |
| Dimethicone | 1.0 |
| Antioxidant | qs |
| Deionized water | to 100 |
| Carbomer, 2% Aqueous Solution | 5.0 |
| Glycerin | 4.0 |
| Potassium Hydroxide | to pH 7.0-7.5 |
| Perfume, Preservative, Colour | qs |

Another cosmeceutical composition within the scope of the invention was prepared. A base formulation in Table 15 was prepared by heating the oil phase and water phase separately to 65° C. to 70° C. The water phase was added to the oil phase with stirring and triethanolamine was at 55° C. to 60° C. The Cromoist O25 was added at 35° C. to 40° C. and then stirred to cool

TABLE 15
Daily Moisturising Cream with Polyunsaturated Fatty Acids

| Component | Percentage by Weight |
|---|---|
| Dioctyl Succinate | 6.0 |
| Caprylic/Capric Triglyceride | 5.0 |
| Glyceryl Stearate | 4.0 |
| Cetearyl Alcohol (and) | 4.0 |
| Ceteareth-20 | |
| Buglossoides arvensis | 3.0 |
| C10-30 Cholesterol/Lanostero | 1.0 |
| BHT | 0.05 |
| Deionized water | to 100 |
| Carbomer, 2% Aqueous Solution | 10.0 |
| Triethanolamine | 0.2 |
| Perfume, Preservative, Colour | qs |
| Aqua (and) Hydrolyzed Oats, such | 2.0 |
| as CROMOIST O25 | |

Another cosmeceutical composition within the scope of the invention was prepared. A base formulation in Table 16 by heating the oil phase and the water phase to 65° C. The water phase was added to the oil phase with stirring. The pH was adjusted to 6.5 to 7.0 with triethanolamine and then stirred to cool.

TABLE 16
After-sun Cream with Polyunsaturated Fatty Acids

| Component | Percentage by Weight |
|---|---|
| Paraffinum Liquidum | 10.0 |
| Nonionic Emulsifying Wax | 3.7 |
| Glyceryl Stearate (and) PEG-100 Croda Stearate | 3.7 |
| Buglossoides arvensis | 3.0 |
| Isopropyl Myristate | 1.0 |
| Menthol | 0.1 |
| BHT | 0.05 |
| Deionized water | to 100 |
| Carbomer, 2% aqueous solution | 7.0 |
| Glycerin | 2.0 |
| Chamomilla recutita extract | 1.0 |
| Triethanolamine | pH 6.5 to 7.0 |
| Perfume, Preservative, Color | qs |

### Example 9

### Use of the Present Invention in Medicines, Foods and Other Oral Preparations

The composition of the invention can be mixed with, as necessary, substances commonly used for medicines, foods and oral preparations. Examples of such substances include other pharmaceutically effective substances, nutrients, animal and plant components, excipients, extenders, sweeteners, flavoring agents, coloring agents, preservatives, emulsifiers, solubilizing agents, hydrotropes and the like. The composition of the present invention can also be taken as a food composition in the form of candies, chewing gums, gummy candies and chewable tablets.

### Example 10

### Production of Adult Dietary Foods and Food Products Using the Present Invention

A preferred composition of the present invention is for use in adult foods and food products. The oil of the present invention can be can be easily mixed in, used in an emulsion or used in a micro-emulsion. When the composition of the invention is taken as a food, it can be used in various liquids, syrups, powders, jellies and the like, by conventional methods. Specific examples of such forms include soft drinks, juice, tea and like beverages (ampuled liquid medicines); powdered juice, powdered soup and like powdered beverages; cookies, biscuits, cereals, chewable tablets, chewing gums, candies and like confections; dressing, sauce, powdered seasoning and like seasonings; and bread, noodles and like staple food products. A food composition of the present invention can be also used as a foodstuffs (e.g., a food additive) to prepare any food. When used as a foodstuff, the food products containing the present invention can be added to a food preparation, for example, commercially available beverages such as milk and the like.

### Example 11

### Production of Geriatric Nutritional Food Products Using the Present Invention

A preferred composition of the present invention is for use in geriatric food products. To create nutritional value for people of advanced age (e.g., the geriatric population), a formula in the composition of a powder, liquid, solid, semi-solid, gel or tablet (e.g., chewable, gelatin-coated or hard) is supplemented with the oil of the present invention.

### Example 12

### Production of Infant Nutritional Food Products Using the Present Invention

A preferred composition of the present invention is for use in infant food products. Liquid, semi-solid, solid and powder food or nutritional products can be prepared using the oil of the present invention and can be manufactured by generally conventional techniques known to those skilled in the art. The liquids may include water, fruit juices such as apple juice, grape juice, orange juice, and the like and vegetable juices such as carrot juice, beet juice, celery juice, tomato juice and the like. Semi-solid baby-food compositions of the invention can contain other ingredients that enhance the acceptability of the composition to an infant. For example, fruit(s) and/or vegetable(s), including purees and juices thereof can also enhance the taste or flavor acceptability of the composition. Solid baby-food products can include such foods as cereals and biscuits while powdered compositions can include powdered baby formula, where these food products are fortified with the oil of the present invention.

### Example 13

### Production of Pet and Aquaculture Food Products Using the Present Invention

A preferred composition of the present invention is for use in pet food products and aquaculture food products. Pet foods for dogs and cats, for example, are usually classified into dry type, semi-moist, soft dry type, wet type, liquid and the like. These pet foods can be supplemented with the oil of the present invention.

In addition, the present invention is useful in production of food products for use in aquaculture. The present invention can be fed to live organisms including but not limited to, shrimp which may be fed to other aquatic organisms or used in other food products, including but not limited to, flakes, pellets or the like.

### Example 14

### Production of Pharmaceutical Products Containing the Present Invention

A preferred composition of the present invention is for pharmaceutical use in a drinking solution, hard capsules or in soft gel capsules. When the composition of the present invention is prepared in the form of a hard capsule or soft gel capsule, the composition may be encapsulated in a gelatin shell which contains any conventional plasticizer. Suitable plasticizers include but are not limited to glycerine, sorbitol, hexanetriol propylene carbonate, hexane glycol, sorbitans, tetrahydrofuryl alcohol ether, diethylene glycol monoethyl ether, 1,3-trimethyl-2-imidazolidone, dimethylisosorbide, and mixtures of these. Encapsulation can be achieved by standard techniques which are well known in the art. A preferred composition of the present invention is a hard capsule or soft gel capsule with 0.1 g to 2 g of the oil of the present invention. Another example of a formulation consists of an emulsion containing from 5% to 80% of the oil of the present invention by weight, where said emulsification can be achieved by standard techniques which are well-known in the art.

What is claimed is:

1. A composition comprising in the range of from 0.1% to 100% by weight of the total composition of an oil extracted from the seeds of Boraginaceae, which oil comprises in the range of from about at least 14.0% to about 17% or greater stearidonic acid and about 7% or lower of gamma-linolenic acid, by weight of the total fatty acids in the oil extract and optionally a physiologically acceptable carrier thereof.

2. A composition according to claim 1, wherein the oil is an extract from seeds of the group consisting of Buglossoides species.

3. A composition according to claim 1, in a form suitable for nutritional use.

4. A composition according to claim 1, wherein the oil is added to food products.

5. A composition according to claim 1, wherein the oil is in the form of a nutritional supplement.

6. A composition according to claim 1, wherein the oil is added to pet food products or aquaculture products.

7. A composition according to claim 1, in a form suitable for cosmetic or cosmeceutical use.

8. A composition according to claim 1, in a form suitable for pharmaceutical use.

9. A composition according to claim 1, in solid form.

10. A composition according to claim 1, in liquid form.

11. A composition according to claim 1, in gel form.

12. A composition according to claim 1, in a hard capsule or soft gel capsule.

13. A composition according to claim 1, wherein the oil is an extract from seeds of Buglossoides arvensis.

14. A composition according to claim 13, in a form suitable for nutritional use.

15. A composition according to claim 13, wherein the oil is added to food products.

16. A composition according to claim 13, wherein the oil is in the form of a nutritional supplement.

17. A composition according to claim 13, wherein the oil is added to pet food products or aquaculture products.

18. A composition according to claim 13, in a form suitable for cosmetic use.

19. A composition according to claim 13, in a form suitable for pharmaceutical use.

20. A composition according to claim 13, in solid form.

21. A composition according to claim 13, in liquid form.

22. A composition according to claim 13, in gel form.

23. A composition according to claim 13, in a hard capsule or soft gel capsule.
